(19) United States
(12) Patent Application Publication
(10) Pub. No.: US 2004/0142929 A1
(43) Pub. Date: Jul. 22, 2004

(54) Derivatives of aryl (or heteroaryl) azolylcarbinoles for the treatment of urinary incontinence

(76) Inventors: Ramon Merce-Vidal, Barcelona (ES); Blas Andaluz-Mataro, Barcelona (ES); Jordi Frigola-Constansa, Barcelona (ES)

Correspondence Address: OSTROLENK FABER GERB & SOFFEN, 1180 AVENUE OF THE AMERICAS, NEW YORK, NY, 100368403

(21) Appl. No.: 10/753,161

(22) Filed: Jan. 6, 2004

Related U.S. Application Data
(63) Continuation-in-part of application No. PCT/ES02/00326, filed on Jul. 1, 2002.
(63) Continuation-in-part of application No. 10/189,915, filed on Jul. 3, 2002.

(30) Foreign Application Priority Data
Jul. 6, 2001 (ES) P 200101587

(51) Int. Cl. A61K 31/5377; A61K 31/454; A61K 31/4196; A61K 31/4178; A61K 31/416

(52) U.S. Cl. 514/235.2; 514/383; 514/396; 514/397; 514/406; 514/326

(57) ABSTRACT

Derivatives of aryl(or heteroaryl)azolylcarbinols of general formula (I), in which Ar represents a phenyl radical or a thienyl radical, optionally substituted, R₁ represents a hydrogen atom or a lower alkyl group, R₂ represents a dialkylaminoalkyl or azaheterocylclylalkyl and Het represents an azole unsubstituted or optionally substituted by one or two substituents, and their physiologically acceptable salts; are useful as drugs in human and/or veterinary therapeutics to treat urinary incontinence in mammals, including man.

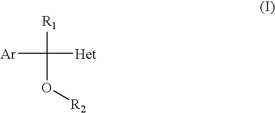

### FIELD OF THE INVENTION

The present invention refers to the use of derivatives of aryl (or heteroaryl) azolylcarbinols of general formula (I), and their physiologically acceptable salts, as medicinal products for human and/or animal therapeutics for the treatment of urinary incontinence.

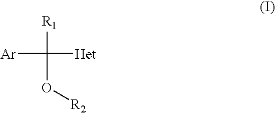

### BACKGROUND OF THE INVENTION

Urination is a function of the lower urinary tract that is defined as discharge of urine through the urethra. Urination is considered to be normal in an adult when it is voluntary, continuous, complete, satisfactory, interruptible, spaced out in time (at socially acceptable intervals), without causing abdominal pressure, without urgency, and only occasional at night.

Urinary incontinence, a urinary disorder, is defined as the involuntarily discharge of urine, which can be demonstrated objectively. This functional disorder of bladder is a health problem of increasing social and hygienic relevance for the population that suffers from it. According to our data, urinary incontinence occurs in approximately 1.5 to 5% of men and 10 to 30% of women in the population between 15 and 64 years old. However, if we select the non-hospitalised population sector over 60 years old, the prevalence ranges from 15% to 35% of this population. On the other hand, when hospitalised patients over 60 years old are studied, the incidence is higher. Urinary incontinence affects approximately 2 million of the Spanish population.

Urinary incontinence can be considered as a symptom, sign or pathological condition. The following is one of the possible classifications of this functional disorder.

Imperative micturition or urge incontinence. This is when the involuntary discharge of urine is accompanied by an intense desire to urinate (urgency). This can be separated into motor urgency incontinence or sensitive urgency incontinence. Motor urgency incontinence is associated with hyperactivity of the detrusor muscle and/or reduced distensibility of the detrusor. Hyperactivity is characterised by involuntary contractions of the detrusor during the filling stage, either spontaneous or provoked, that the patient cannot totally suppress. Hyperactivity of the detrusor muscle can occur when there is obstruction of the exiting urinary flow, inflammation and conditions in which the bladder is irritated, or it can be of unknown aetiology (idiopathic).

Hyperreflexia, is described as a condition that presents uncontrolled contractions of the detrusor muscle associated with neurological disorders such as multiple sclerosis or plaque sclerosis, sequelae of medular traumatisms or Parkinson's disease.

Urinary stress incontinence due to a defective urethral closure mechanism, there is involuntary discharge of urine in the absence of detrusor contraction that occurs when the intravesical pressure exceeds the pressure in the urethra. Involuntary discharge occurs when some physical exertion is made such as jumping, coughing, going down stairs etc. One additional factor can be due to structural changes in the urethra due to menopausal hypooestrogenia.

Mixed incontinence, this term refers to the existence of both urgency incontinence and stress incontinence.

Enuresis, this term refers to any involuntary loss of urine and more specifically refers to incontinence during sleep. It most often applies to children with a higher incidence in boys and in the age group of up to 5 years.

For more definitions and a standardisation of Terminology reference is made to Abrams et al, Neurology and Urodynamics 21:167-178 (2002)

The therapeutic options for urinary incontinence depend on the type of incontinence. In urgency incontinence, the first and most effective therapeutic approach is pharmacological treatment accompanied by a series of hygiene regulations and patient education, with secondary approaches including other therapies such as maximum electrical stimulation or surgical treatment. Conservative measures such as pelvic floor exercises and surgical treatment, as a first option, are reserved for stress incontinence.

Pharmacological treatment of urinary urgency incontinence and of hyperreflexia is aimed at reducing activity of the detrusor muscle and increasing the bladder capacity. In cases of stress incontinence, the treatment is aimed at increasing resistance to urinary discharge.

The drugs used to treat urinary incontinence include a wide therapeutic range of drugs from different pharmacological groups with different action mechanisms [Hattori T., Drug treatment of urinary incontinence. Drugs of Today, 1998, 34 (2): 125-138], although there is a great deal of confusion and the clinical efficacy of these has not been completely demonstrated.

In a first group of drugs that have an anticholinergic action, propantheline can be considered as a pure anticholinergic agent. There is also a new drug, tolterodine, that has a selective anticholinergic action but that is not selective for the different subtypes of muscarinic receptors although it does appear to have a selectivity of action that is centred around the urinary bladder (detrusor), salivary glands and human intestine. One of the drugs with an anticholinergic action, oxybutin, is a drug with a mixed action, a moderate anticholinergic agent and is a strong direct muscular relaxant. Oxybutin is now the first drug of choice for this disorder, in spite of its tolerability profile with non-severe but annoying adverse effects such as dry mouth, constipation and drowsiness that, in some cases, can cause the patient to abandon the treatment.

Several tricyclic antidepressants have beneficial effects in patients with detrusor hyperactivity. Imipramine, a drug used in clinical practise, has been shown to be an effective treatment for nocturnal enuresis in children and vesical hyperactivity, for example, in the elderly. Owing to the different adverse events reported for this group of drugs, sometimes of strong intensity (e.g. cardiovascular events), the risk-benefits of this treatment for urination disorders must be studied in certain populations, especially in the elderly.

The α-adrenergic antagonists such as prazosin, terazosin or doxazosin can improve detrusor hyperactivity and symptoms related with detrusor dysfunction in patients with benign prostrate hyperplasia, although the evidence for this effect in hyperactive bladder is currently under discussion and there are no data to support its use in urgency incontinence.

Another therapeutically interesting group corresponds to the β-adrenergics, although there is still little information available about their efficacy. It is known that β-adrenergic stimulation can relax the human bladder in normal conditions. The detrusor muscle, both in normal conditions or in the case of an unstable bladder shows a similar degree of response, relaxation, to an p-agonist drug. The β₂-adrenergic receptor agonists, such as terbutaline or albuterol, have been shown to be able to increase the bladder capacity. In contrast, efficacy of this drug in the treatment of detrusor hyperactivity has been shown in very few controlled clinical studies and in only a small sample of patients.

In our patents EP 289380 B1 (U.S. Pat. No. 5,017,596) and WO 99/52525 (U.S. Pat. No. 6,410,582) we have described derivatives of carbinols of general formula (I) with analgesic activity,

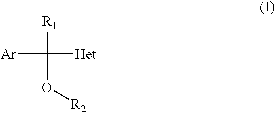

In these compounds of general formula (I). Ar represents a benzene ring or a thiophene ring with or without substitutions, R₁ represents a hydrogen atom or a lower alkyl group from C₁ to C₄; R₂ represents a dialkylaminoalkyl or azaheterocyclylalkyl and Het represents an azole with or without substitutions, and their physiologically acceptable salts.

In our patent applications WO 97/20817 (U.S. Pat. No. 5,849,931), WO 99/02500 (U.S. Pat. No. 6,187,930), WO 99/07684 (U.S. Pat. No. 6,118,009) and WO 99/52525 (U.S. Pat. No. 6,410,582) we have also described several procedures to prepare enantiomerically pure compounds with general formula (I).

We have also discovered now that general formula (I) compounds, and their physiologically acceptable salts, are especially useful for producing drugs, in human or veterinary therapeutics, to cure or relieve urinary incontinence.

### DETAILED DESCRIPTION OF THE INVENTION

The present invention refers to the use of derivatives of aryl (or heteroaryl) azolylcarbinols of general formula (I)

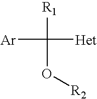

in which

Ar represents a phenyl radical or a thienyl radical, without substitutions or optionally with 1,2 or 3 equal or different substituents selected from a group comprised of fluorine, chlorine, bromine, methyl, trifluoromethyl and methoxy;

R₁ represents a hydrogen atom or a lower alkyl group from C₁ to C₄;

R₂ represents a dialkyl (C₁-C₄) aminoalkyl (C₂-C₃) radical, or azaheterocyclylalkyl (C₂-C₃); and

Het represents an azole, i.e. a five-membered nitrogenated aromatic heterocycle that contains from one to three nitrogen atoms, without substitutions or optionally with substitutions by 1 or 2 equal or different substituents selected from a group comprised of fluorine, chlorine, bromine and methyl;

or one of its physiologically acceptable salts,

In the production of a drug to treat urinary incontinence, in mammals, including man, especially in patients that present an urgency or hyperreflexive incontinence.

The term “lower alkyl group from C₁ to C₄” (which is equivalent to “lower C₍₁₋₄)-Alkyl”) represents a linear or branched chain radical derived from a saturated hydrocarbon of 1 to 4 carbon atoms, such as methyl, ethyl, propyl, isopropyl, butyl, isobutyl, sec-butyl and tert-butyl.

The term “dialkyl(C₁-C₄)aminoalkyl (C₂-C₃), or azaheterocyclylalkyl (C₂-C₃)” represents an alkyl radical with two or three carbon atoms joined to a dialkyl (C₁-C₄) amine or to a cyclic amine, such as, for example, dimethylaminoethyl, dimethylaminopropyl, diethylaminoethyl, piperidinylethyl, morpholinylpropyl, pirrolidinylalkyl, etc.

Illustrative examples of compounds included in the present invention include:

(±)-5-{α-[2-(dimethylamino)ethoxy]benzyl}-1-methyl-1H-pirazole.

(±)-5-{α-[2-(dimethylamino)ethoxy]benzyl}-1-methyl-1H-pirazole citrate.

(+)-5-{α-[2-(dimethylamino)ethoxy]benzyl}-1-methyl-1H-pirazole.

(−)-5-{α-[2-(dimethylamino)ethoxy]benzyl}-1-methyl-1H-pirazole.

(+)-5-{α-[2-(dimethylamino)ethoxy]benzyl}-1-methyl-1H-pirazole citrate.

(−)-5-{α-[2-(dimethylamino)ethoxy]benzyl}-1-methyl-1H-pirazole.

(±)-5-{α-[2-(dimethylamino)ethoxy]-2-thienylmethyl}-1-methyl-1H-pirazole.

(±)-5-{α-[2-(dimethylamino)ethoxy]-2-thienylmethyl}-1-methyl-1H-pirazole citrate.

(+)-5-{α-[2-(dimethylamino)ethoxy]-2-thienylmethyl}-1-methyl-1H-pirazole.

(−)-5-{α-[2-(dimethylamino)ethoxy]-2-thienylmethyl}-1-methyl-1H-pirazole.

(+)-5-{α-[2-(dimethylamino)ethoxy]-2-thienylmethyl}-1-methyl-1H-pirazole citrate.

(−)-5-{α-[2-(dimethylamino)ethoxy]-2-thienylmethyl}-1-methyl-1H-pirazole citrate.

The compounds of general formula (I) can be synthesised according to the procedures described in patents EP 289380, U.S. Pat. No. 5,017,596 or WO 99/52525. The compounds of general formula (I) have a stereogenic centre and the invention refers both to the use of a pure enantiomer and to the use of a mixture of enantiomers. The enantiomers can be prepared by any of the procedures described in our patents WO 97/20817 (U.S. Pat. No. 5,849,931), WO 99/02500 (U.S. Pat. No. 6,187,930), WO 99/07684 (U.S. Pat. No. 6,118,009) and WO 99/52525 (U.S. Pat. No. 6,410,582).

In the context of this invention the term “(dimethylamino)” shall be treated and considered absolutely identical to the term “(dimethylamine). The selection of the first term to describe compounds was only due a seeming more fitting chemical nomenclature.

In the present invention, the activity of general formula (I) compounds has been demonstrated in processes of hyperactivity of the urinary bladder, and they are, therefore, useful in urinary incontinence due to hyperreflexive detrusor activity and urgency incontinence. The same applies to enuresis or stress incontinence. Another aspect of the invention is a method of treatment of a patient or a mammal, including man, suffering from urinary incontinence characterized in that the method comprises the administration of a therapeutically effective amount of a compound of general formula (I)

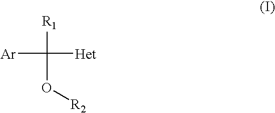

in which

Ar represents a phenyl radical or a thienyl radical, with no substitutions or optionally with 1, 2 or 3 equal or different substituents, selected from a group consisting of fluoride, chloride, bromide, methyl, trifluoromethyl and methoxy;

R₁ represents hydrogen or a lower alkyl group from C₁ to C₄;

R₂ represents a dialkyl(C₁-C₄)aminoalkyl (C₂-C₃), or azaheterocyclylalkyl (C₂-C₃) radical; and

Het represents a five-armed nitrogenated aromatic heterocycle that contains one to three nitrogen atoms, without substitutions or optionally substituted by 1 or 2 equal or different substituents selected from a group consisting of fluoride, chloride, bromide and methyl;

optionally in the form of its racemate, pure stereoisomers, especially enantiomers or diastereomers or in the form of mixtures of stereoisomers, especially enantiomers or diastereomers, in any suitable ratio;

in the form shown or in form of the acid or base or in form of a salt, especially a physiologically acceptable salt, or in form of a solvate, especially a hydrate.

The term “salt” is to be understood as meaning any form of the active compound according to the invention in which this assumes an ionic form or is charged and is coupled with a counter-ion (a cation or anion) or is in solution. By this are also to be understood complexes of the active compound with other molecules and ions, in particular complexes which are complexed via ionic interactions.

The term “physiologically acceptable salt” is understood in particular, in the context of this invention, as salt formed either with a physiologically tolerated acid, that is to say salts of the particular active compound with inorganic or organic acids which are physiologically tolerated—especially if used on humans and/or mammals—or with at least one, preferably inorganic, cation which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid, citric acid, glutamic acid, 1,1-dioxo-1,2-dihydrol6-benzo[d]isothiazol-3-one (saccharin acid), monomethylsebacic acid, 5-oxo-proline, hexane-1-sulfonic acid, nicotinic acid, 2-, 3- or 4-aminobenzoic acid, 2,4,6-trimethyl-benzoic acid, alpha-lipoic acid, acetylglycine, acetylsalicylic acid, hippuric acid and/or aspartic acid. Examples of physiologically tolerated salts of particular bases are salts of alkali metals and alkaline earth metals and with NH₄.

In the context of this invention the preferred salt is a salt of the particular active compound with a physiologically tolerated acid.

The salt particularly preferred in the context of this invention is the citrate.

In the context of this invention patient does mean any human being in need of treatment. In particular this encompasses man, woman and children. Depending on the specific kind of Urinary Incontinence encountered, the patient group most preferably treated can vary and at times (for example with stress incontinence) include more women, sometimes more elderly women, at times more men, especially elderly men, and sometimes, more children (for example in enuresis).

A preferred method according to the invention is characterized in that it comprises the administration of a compound of general formula (I), in which R₁ is selected from hydrogen or from a group consisting of methyl, ethyl, propyl, isopropyl, butyl, isobutyl, sec-butyl and tert-butyl.

Another preferred method according to the invention is characterized in that it comprises the administration of a compound of general formula (I), in which R₂ is selected from among a group consisting of dimethylaminoethyl, dimethylaminopropyl, diethylaminoethyl, piperidinylethyl, morpholinylpropyl and pirrolidinylethyl.

Another preferred method according to the invention is characterized in that it comprises the administration of a compound of general formula (Ia)

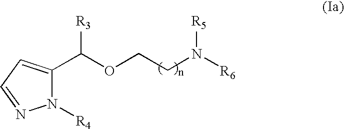

in which

n is 1 or 2;

R₃ is selected from:

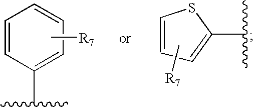

R₄ is selected from hydrogen, fluoride, chloride, bromide and methyl;

R₅ and R₆ are independently selected from lower C₍₁₋₄₎-Alkyl or together with the Nitrogen form an azaheterocyclic ring;

R₇ is selected from the group consisting of hydrogen, fluoride, chloride, bromide, methyl, trifluoromethyl and methoxy.

A preferred method according to (this (above) aspect of) the invention is characterized in that it comprises the administration of a compound of general formula (Ia), in which R₇ is hydrogen.

Another preferred method according to (this (above) aspect of) the invention is characterized in that it comprises the administration of a compound of general formula (Ia), in which R₄ is Methyl.

Another preferred method according to (this (above) aspect of) the invention is characterized in that it comprises the administration of a compound of general formula (Ia), in which R₄ and R₅ are either CH₃ or C₂H₅ or together with the Nitrogen form a piperidinyl, morpholinyl or pirrolidinyl ring.

Another preferred method according to (this (above) aspect of) the invention is characterized in that it comprises the administration of a compound of general formula (Ia) selected from among a group consisting of:

5-{α-[2-(dimethylamino)ethoxy]benzyl}-1-methyl-1H-pirazole,

(±)-5-{α-[2-(dimethylamino)ethoxy]benzyl}-1-methyl-1H-pirazole,

(+)-5-{α-[2-(dimethylamino)ethoxy]benzyl}-1-methyl-1H-pirazole,

(−)-5-{α-[2-(dimethylamino)ethoxy]benzyl}-1-methyl-1H-pirazole,

5-{α-[2-(dimethylamino)ethoxy]benzyl}-1-methyl-1H-pirazole citrate,

(+)-5-{α-[2-(dimethylamino)ethoxy]benzyl}-1-methyl-1H-pirazole citrate,

(+)-5-{α-[2-(dimethylamino)ethoxy]benzyl}-1-methyl-1H-pirazole citrate,

(−)-5-{α-[2-(dimethylamino)ethoxy]benzyl}-1-methyl-1H-pirazole citrate,

5-{α-[2-(dimethylamino)ethoxy]-2-thienylmethyl}-1-methyl-1H-pirazole,

(+)-5-{α-[2-(dimethylamino)ethoxy]-2-thienylmethyl}-1-methyl-1H-pirazole,

(+)-5-{α-[2-(dimethylamino)ethoxy]-2-thienylmethyl}-1-methyl-1H-pirazole,

(−)-5-{α-[2-(dimethylamino)ethoxy]-2-thienylmethyl}-1-methyl-1H-pirazole,

5-{α-[2-(dimethylamino)ethoxy]-2-thienylmethyl}-1-methyl-1H-pirazole citrate,

(+)-5-{α-[2-(dimethylamino)ethoxy]-2-thienylmethyl}-1-methyl-1H-pirazole citrate,

(+)-5-{α-[2-(dimethylamino)ethoxy]-2-thienylmethyl}-1-methyl-1H-pirazole citrate,

(−)-5-{α-[2-(dimethylamino)ethoxy]-2-thienylmethyl}-1-methyl-1H-pirazole citrate.

Another preferred method according to (this (above) aspect of) the invention is characterized in that it comprises the administration of a compound of general formula (Ib)

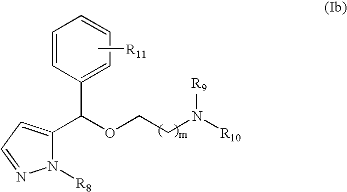

in which

m is 1 or 2;

R₈ is selected from hydrogen, fluoride, chloride, bromide and methyl;

R₉ and R₁₀ are independently selected from lower C₍₁₋₄₎-Alkyl or together with the Nitrogen form an azaheterocyclic ring;

R₁₁ is selected from the group consisting of hydrogen, fluoride, chloride, bromide, methyl, trifluoromethyl and methoxy.

A preferred method according to (this (above) aspect of) the invention is characterized in that it comprises the administration of a compound of general formula (Ib), in which R₁₁ is hydrogen.

Another preferred method according to (this (above) aspect of) the invention is characterized in that it comprises the administration of a compound of general formula (Ib), in which R₈ is Methyl.

Another preferred method according to (this (above) aspect of) the invention is characterized in that it comprises the administration of a compound of general formula (Ib), in which R₉ and R₁₀ are either CH₃ or C₂H₅ or together with the Nitrogen form a piperidinyl, morpholinyl or pirrolidinyl ring;

preferably in which R₉ and R₁₀ are either CH₃ or C₂H₅;

especially in which R₉ and R₁₀ are equal and either CH₃ or C₂H₅;

most preferably in which R₉ and R₁₀ are both CH₃.

Another preferred method according to (this (above) aspect of) the invention is characterized in that it comprises the administration of a compound of general formula (Ib), in which m is 1.

Another preferred method according to (this (above) aspect of) the invention is characterized in that it comprises the administration of a compound of general formula (Ib) selected from among a group consisting of:

5-{α-[2-(dimethylamino)ethoxy]benzyl}-1-methyl-1H-pirazole,

(+)-5-{α-[2-(dimethylamino)ethoxy]benzyl}-1-methyl-1H-pirazole,

(+)-5-{α-[2-(dimethylamino)ethoxy]benzyl}-1-methyl-1H-pirazole,

(−)-5-{α-[2-(dimethylamino)ethoxy]benzyl}-1-methyl-1H-pirazole,

5-{α-[2-(dimethylamino)ethoxy]benzyl}-1-methyl-1H-pirazole citrate,

(±)-5-{α-[2-(dimethylamino)ethoxy]benzyl}-1-methyl-1H-pirazole citrate,

(+)-5-{α-[2-(dimethylamino)ethoxy]benzyl}-1-methyl-1H-pirazole citrate,

(−)-5-{α-[2-(dimethylamino)ethoxy]benzyl}-1-methyl-1H-pirazole citrate.

Another preferred method according to (this (above) aspect of) the invention is characterized in that it comprises the administration of

5-{α-[2-(dimethylamino)ethoxy]benzyl}-1-methyl-1H-pirazole.

Another preferred method according to (this (above) aspect of) the invention is characterized in that it comprises the administration of

(±)-5-{α-[2-(dimethylamino)ethoxy]benzyl}-1-methyl-1H-pirazole.

Another preferred method according to (this (above) aspect of) the invention is characterized in that it comprises the administration of

(+)-5-{α-[2-(dimethylamino)ethoxy]benzyl}-1-methyl-1H-pirazole.

Another preferred method according to (this (above) aspect of) the invention is characterized in that it comprises the administration of

(−)-5-{α-[2-(dimethylamino)ethoxy]benzyl}-1-methyl-1H-pirazole.

Another preferred method according to (this (above) aspect of) the invention is characterized in that it comprises the administration of

5-{α-[2-(dimethylamino)ethoxy]benzyl}-1-methyl-1H-pirazole citrate.

Another preferred method according to (this (above) aspect of) the invention is characterized in that it comprises the administration of

(+)-5-{α-[2-(dimethylamino)ethoxy]benzyl}-1-methyl-1H-pirazole citrate.

Another preferred method according to (this (above) aspect of) the invention is characterized in that it comprises the administration of

(+)-5-{α-[2-(dimethylamino)ethoxy]benzyl}-1-methyl-1H-pirazole citrate.

Another preferred method according to (this (above) aspect of) the invention is characterized in that it comprises the administration of

(−)-5-{α-[2-(dimethylamino)ethoxy]benzyl}-1-methyl-1H-pirazole citrate.

A preferred method according to the invention is characterized in that it comprises the administration of a compound of general formula (Ic)

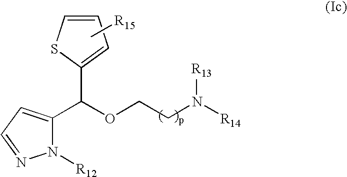

in which

p is 1 or 2;

R₁₂ is selected from hydrogen, fluoride, chloride, bromide and methyl; R₁₃ and R₁₄ are independently selected from lower C₍₁₋₄₎-Alkyl or together with the Nitrogen form an azaheterocyclic ring;

R₁₅ is selected from the group consisting of hydrogen, fluoride, chloride, bromide, methyl, trifluoromethyl and methoxy.

A preferred method according to (this (above) aspect of) the invention is characterized in that it comprises the administration of a compound of general formula (Ic), in which R₁₅ is hydrogen.

Another preferred method according to (this (above) aspect of) the invention is characterized in that it comprises the administration of a compound of general formula (Ic), in which R₁₂ is Methyl.

Another preferred method according to (this (above) aspect of) the invention is characterized in that it comprises the administration of a compound of general formula (Ic), in which R₁₃ and R₁₄ are either CH₃ or C₂H₅ or together with the Nitrogen form a piperidinyl, morpholinyl or pirrolidinyl ring;

preferably in which R₁₃ and R₁₄ are either CH₃ or C₂H₅;

especially in which R₁₃ and R₁₄ are equal and either CH₃ or C₂H₅;

most preferably in which R₁₃ and R₁₄ are both CH₃.

Another preferred method according to (this (above) aspect of) the invention is characterized in that it comprises the administration of a compound of general formula (Ic), in which p is 1.

Another preferred method according to (this (above) aspect of) the invention is characterized in that it comprises the administration of a compound of general formula (Ic) selected from among a group consisting of:

5-{α-[2-(dimethylamino)ethoxy]-2-thienylmethyl}-1-methyl-1H-pirazole,

(±)-5-{α-[2-(dimethylamino)ethoxy]-2-thienylmethyl}-1-methyl-1H-pirazole,

(+)-5-{α-[2-(dimethylamino)ethoxy]-2-thienylmethyl}-1-methyl-1H-pirazole,

(−)-5-{α-[2-(dimethylamino)ethoxy]-2-thienylmethyl}-1-methyl-1H-pirazole,

5-{α-[2-(dimethylamino)ethoxy]-2-thienylmethyl}-1-methyl-1H-pirazole citrate,

(±)-5-{α-[2-(dimethylamino)ethoxy]-2-thienylmethyl}-1-methyl-1H-pirazole citrate,

(+)-5-{α-[2-(dimethylamino)ethoxy]-2-thienylmethyl}-1-methyl-1H-pirazole citrate,

(−)-5-{α-[2-(dimethylamino)ethoxy]-2-thienylmethyl}-1-methyl-1H-pirazole citrate.

Another preferred method according to (this (above) aspect of) the invention is characterized in that it comprises the administration of:

5-{α-[2-(dimethylamino)ethoxy]-2-thienylmethyl}-1-methyl-1H-pirazole,

,optionally in the form of its racemate, pure stereoisomers, especially enantiomers or diastereomers or in the form of mixtures of stereoisomers, especially enantiomers or diastereomers, in any suitable ratio;

in form of the free base or in form of a salt, especially a physiologically acceptable salt, or in form of a solvate, especially a hydrate.

Another preferred method according to (this (above) aspect of) the invention is characterized in that it comprises the administration of:

5-{α-[2-(dimethylamino)ethoxy]-2-thienylmethyl}-1-methyl-1H-pirazole citrate,

,optionally in the form of its racemate, pure stereoisomers, especially enantiomers or diastereomers or in the form of mixtures of stereoisomers, especially enantiomers or diastereomers, in any suitable ratio.

A preferred method according to the invention is characterized in that man means a female.

A preferred method according to the invention is characterized in that man means a male.

A preferred method according to the invention is characterized in that the patient is a woman.

A preferred method according to the invention is characterized in that the patient is an elderly woman.

A preferred method according to the invention is characterized in that the patient is a man.

25

A preferred method according to the invention is characterized in that the patient is an elderly man.

A preferred method according to the invention is characterized in that the patient is a child. A preferred method according to the invention is characterized in that the urinary incontinence the patient or mammal, including man, is suffering from is urge urinary incontinence.

A preferred method according to the invention is characterized in that the urinary incontinence the patient or mammal, including man, is suffering from is stress urinary incontinence or urinary stress incontinence.

A preferred method according to the invention is characterized in that the urinary incontinence the patient or mammal, including man, is suffering from is hyperreflexive urinary incontinence.

A preferred method according to the invention is characterized in that the urinary incontinence the patient or mammal, including man, is suffering from is enuresis.

A preferred method according to the invention is characterized in that the therapeutically effective amount of the active compound is administered at a dose between 50 and 400 mg/day or between 200 and 600 mg/day.

In the context of this invention—if not clearly expressed as meaning the complete salt—dose means the dose of the active compound without the salt (which means without the counter ion, for example the citrate ion).

A preferred method according to the invention is characterized in that the compound is administered in form of a tablet or capsule.

A preferred method according to the invention is characterized in that the compound is administered in form of an immediate release formulation.

In the context of this invention “immediately release formulation” means any formulation with a release profile from which measured according to a standard measurement (e.g. using the paddle method according to the Pharmacopeia) (e.g. in 0.1% NaCl solution) within 30 minutes more than 50%, more preferably 60%, or even more preferably 70% of the active compound is released.

A specially preferred aspect is a method of treatment of a patient suffering from urinary incontinence characterized in that the method comprises the administration of a therapeutically effective amount of

5-{α-[2-(dimethylamino)ethoxy]benzyl}-1-methyl-1H-pirazole,

,optionally in the form of its racemate, pure stereoisomers, especially enantiomers or diastereomers or in the form of mixtures of stereoisomers, especially enantiomers or diastereomers, in any suitable ratio;

in form of the free base or in form of a salt, especially a physiologically acceptable salt, or in form of a solvate, especially a hydrate.

A preferred method according to the specially preferred aspect of the invention is characterized in that it comprises the administration of

(+)-5-{α-[2-(dimethylamino)ethoxy]benzyl}-1-methyl-1H-pirazole.

Another preferred method according to the specially preferred aspect of the invention is characterized in that it comprises the administration of

(+)-5-{α-[2-(dimethylamino)ethoxy]benzyl}-1-methyl-1H-pirazole.

Another preferred method according to the specially preferred aspect of the invention is characterized in that it comprises the administration of

(−)-5-{α-[2-(dimethylamino)ethoxy]benzyl}-1-methyl-1H-pirazole.

Another preferred method according to the specially preferred aspect of the invention is characterized in that it comprises the administration of

5-{α-[2-(dimethylamino)ethoxy]benzyl}-1-methyl-1H-pirazole citrate.

Another preferred method according to the specially preferred aspect of the invention is characterized in that it comprises the administration of

(±)-5-{α-[2-(dimethylamino)ethoxy]benzyl}-1-methyl-1H-pirazole citrate.

Another preferred method according to the specially preferred aspect of the invention is characterized in that it comprises the administration of

(+)-5-{α-[2-(dimethylamino)ethoxy]benzyl}-1-methyl-1H-pirazole citrate.

Another preferred method according to the specially preferred aspect of the invention is characterized in that it comprises the administration of

(−)-5-{α-[2-(dimethylamino)ethoxy]benzyl}-1-methyl-1H-pirazole citrate.

Another preferred method according to the specially preferred aspect of the invention is characterized in that the patient is a woman.

Another preferred method according to the specially preferred aspect of the invention is characterized in that the patient is an elderly woman.

Another preferred method according to the specially preferred aspect of the invention is characterized in that the patient is a man.

Another preferred method according to the specially preferred aspect of the invention is characterized in that the patient is an elderly man.

Another preferred method according to the specially preferred aspect of the invention is characterized in that the patient is a child.

Another preferred method according to the specially preferred aspect of the invention is characterized in that the urinary incontinence the patient is suffering from is urge urinary incontinence.

Another preferred method according to the specially preferred aspect of the invention is characterized in that the urinary incontinence the patient is suffering from is stress urinary incontinence or urinary stress incontinence.

Another preferred method according to the specially preferred aspect of the invention is characterized in that the urinary incontinence the patient is suffering from is hyperreflexive urinary incontinence.

Another preferred method according to the specially preferred aspect of the invention is characterized in that the urinary incontinence the patient is suffering from is enuresis.

Another preferred method according to the specially preferred aspect of the invention is characterized in that the therapeutically effective amount of 5-{α-[2-(dimethylamino)ethoxy]benzyl}-1-methyl-1H-pirazole is administered at a dose between 50 and 400 mg/day or between 200 and 600 mg/day.

Another preferred method according to the specially preferred aspect of the invention is characterized in that the therapeutically effective amount of (±)-5-{α-[2-(dimethylamino)ethoxy]benzyl}-1-methyl-1H-pirazole is administered at a dose between 50 and 400 mg/day or between 200 and 600 mg/day.

Another preferred method according to the specially preferred aspect of the invention is characterized in that the therapeutically effective amount of (+)-5-{α-[2-(dimethylamino)ethoxy]benzyl}-1-methyl-1H-pirazole is administered at a dose between 50 and 400 mg/day or between 200 and 600 mg/day.

Another preferred method according to the specially preferred aspect of the invention is characterized in that the therapeutically effective amount of (−)-5-{α-[2-(dimethylamino)ethoxy]benzyl}-1-methyl-1H-pirazole is administered at a dose between 50 and 400 mg/day or between 200 and 600 mg/day.

Another preferred method according to the specially preferred aspect of the invention is characterized in that 5-{α-[2-(dimethylamino)ethoxy]benzyl}-1-methyl-1H-pirazole is administered at a dose of 230 mg/day, 460 mg/day or 345 mg/day.

Another preferred method according to the specially preferred aspect of the invention is characterized in that (±)-5-{α-[2-(dimethylamino)ethoxy]benzyl}-1-methyl-1H-pirazole is administered at a dose of 230 mg/day, 345 mg/day, 460 mg/day or 575 mg/day, preferably 345 mg/day or 460 mg/day.

Another preferred method according to the specially preferred aspect of the invention is characterized in that 5-{α-[2-(dimethylamino)ethoxy]benzyl}-1-methyl-1H-pirazole citrate is administered at a dose of 400 mg/day, 600 mg/day, 800 mg/day or 1000 mg/day, preferably 600 mg/day or 800 mg/day.

Another preferred method according to the specially preferred aspect of the invention is characterized in that (±)-5-{α-[2-(dimethylamino)ethoxy]benzyl}-1-methyl-1H-pirazole citrate is administered at a dose of 400 mg/day, 600 mg/day, 800 mg/day or 1000 mg/day, preferably 600 mg/day or 800 mg/day.

Another preferred method according to the specially preferred aspect of the invention is characterized in that 5-{α-[2-(dimethylamino)ethoxy]benzyl}-1-methyl-1H-pirazole is administered twice daily.

Another preferred method according to the specially preferred aspect of the invention is characterized in that 5-{α-[2-(dimethylamino)ethoxy]benzyl}-1-methyl-1H-pirazole is administered orally.

Another preferred method according to the specially preferred aspect of the invention is characterized in that 5-{α-[2-(dimethylamino)ethoxy]benzyl}-1-methyl-1H-pirazole is administered in form of a tablet or capsule.

Another preferred method according to the specially preferred aspect of the invention is characterized in that 5-{α-[2-(dimethylamino)ethoxy]benzyl}-1-methyl-1H-pirazole is administered in form of an immediate release formulation.

Another preferred method according to the specially preferred aspect of the invention is characterized in that 5-{α-[2-(dimethylamino)ethoxy]benzyl}-1-methyl-1H-pirazole is administered in form of a formulation comprising any of the following:

sodium croscarmelose

colloidal silica dioxide,

a salt with stearic acid, especially magnesium stearate,

povidone,

microcrystalline cellulose

lactose monohydrate

polyethylene glycol.

Another preferred method according to the specially preferred aspect of the invention is characterized in that 5-{α-[2-(dimethylamino)ethoxy]benzyl}-1-methyl-1H-pirazole is administered in form of a formulation according to example 5.

Another preferred method according to the specially preferred aspect of the invention is characterized in that 5-{α-[2-(dimethylamino)ethoxy]benzyl}-1-methyl-1H-pirazole is administered in form of a formulation according to example 7.

Taking into account its good pharmacodynamic properties, derivatives of aryl(or heteroaryl)azolylcarbinol, according to the invention, can be used satisfactorily in human and animal therapeutics to cure and relieve urinary incontinence.

In human therapeutics, the dose administered of the compounds of the invention depends on the severity of the infection to be treated. It is normally between 50 and 400 mg/day or 200 and 600 mg/day. The compounds of the invention are administered for example in the form of capsules or tablets.

Any formulation or pharmaceutical composition according to the invention contains the active ingredient as well as optionally at least one auxiliary material and/or additive.

The auxiliary material and/or additive can be selected from carrier, excipient, support materials, glidants, fillers, solvents, diluents, colorants, taste conditioners like sugars, antioxidants and/or binders. In the case of a suppository this might involve waxes or fatty acid esters or conserving agents, emulsifiers and/or carriers for parenteral application. The selection of these auxiliary materials and/or additives and of the amounts to be used depends upon how the pharmaceutical composition is to be applied.

Examples include here oral or parenteral like pulmonal, nasal, rectal and/or intravenous application. Therefore the pharmaceutical composition according to the invention can be adapted for topical or systemical application, especially dermal, subcutaneous, intramuscular, intra-articular and/or intraperitoneal, pulmonal, buccal, sublingual, nasal, percutaneous, vaginal, oral or parenteral, pulmonal, nasal, rectal and/or intravenous application.

For oral application preparations in the form of tablets, chewable tablets, dragees, capsules, granules, drops, juices and syrups are suitable. Solutions, suspensions, readily reconstitutable dry preparations and sprays are suitable i.a. for parenteral application. The compounds according to the invention as a deposit in a dissolved form or in a patch, optionally with the addition of agents which promote dermal penetration, are examples of suitable percutaneous forms of application. Dermal applications include i.a. an ointment, a gel, a cream, a lotion, a suspension, an emulsion whereas the preferred form for rectal application is a suppository.

The examples and figures in the following section describing pharmacological trials are merely illustrative and the invention cannot be considered in any way as being restricted to these applications.

Figures:

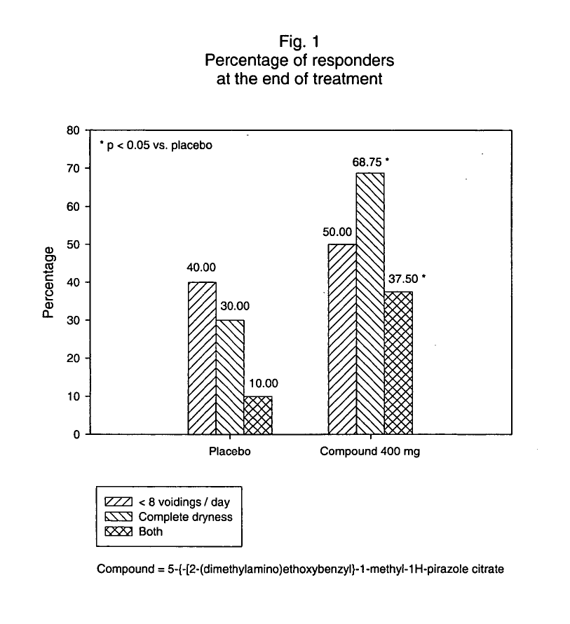

FIG. 1 illustrates results of example 2.

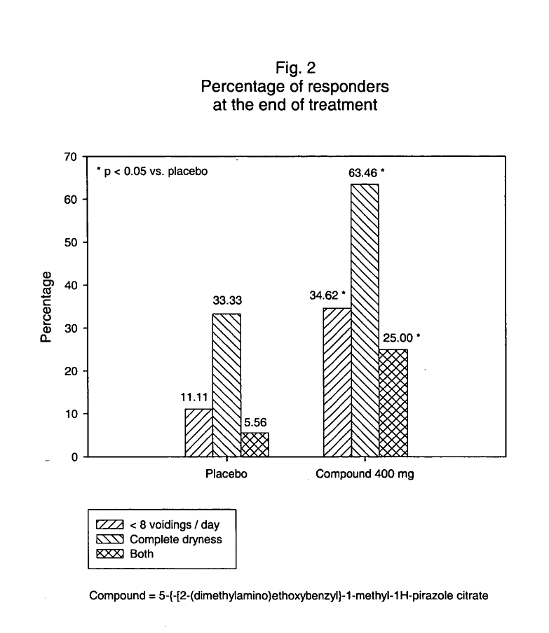

FIG. 2 illustrates results of example 3.

### EXAMPLES

### Example 1

(±)-5-{o-[2-(dimethylamino)ethoxy]benzyl}-1-methyl-1H-pirazole or (±)-5-{α-[2-(dimethylamino)ethoxy]benzyl}-1-methyl-1H-pirazole citrate of the formula

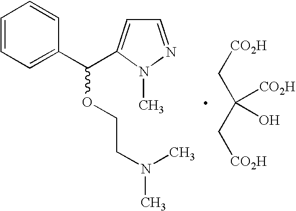

is already described in U.S. Pat. No. 5,017,596 and EP 289 380 B1 including its synthesis and analgetic properties.

### Example 2

Clinical Study A:

In a placebo controlled clinical trial 79 patients were randomised. Patients from both genders aged between 18 and 80 years (both inclusive) were included. They had Urinary incontinence secondary to overactive bladder (detrusor hyperreflexia or instability) or idiopathic urge incontinence confirmed by the medical history and urodynamic study.

The patients were treated within one group with (±)-5-{α-[2-(dimethylamino)ethoxy]benzyl}-1-methyl-1H-pirazole citrate, 400 mg, twice a day, tablets for oral administration, or within another group with placebo in matching tablets, twice a day, for administration by oral route. The patients were treated for 84 days.

Efficacy was measured by the difference from baseline in the mean number of leakages, micturitions, urgencies and voidings/24 hours as provided by a 7-day frequency-volume chart in the end of study visit

The primary efficacy analysis was based on the PP population. The treatment groups were compared with respect to the treatment effect, defined as the difference between treatment groups for changes from baseline in the number of voidings per 24 hours.

Four hundred mg dose of (±)-5-{α-[2-(dimethylamino)ethoxy]benzyl}-1-methyl-1H-pirazole citrate showed a significant reduction of the mean number of daily voidings, leakages, urgencies and micturitions compared with placebo.

As an addition the percentage of responders was compared based on an analysis of the number of patients having <8 voidings/day or experienced complete dryness or both. The statistical significance was determined. (±)-5-{α-[2-(dimethylamino)ethoxy]benzyl}-1-methyl-1H-pirazole was superior to placebo (see FIG. 1).

This clinical trial has demonstrated the potential of (±)-5-{α-[2-(dimethylamino)ethoxy]benzyl}-1-methyl-1H-pirazole citrate 400 mg twice a day. (equivalent to 230 mg of (±)-5-{α-[2-(dimethylamino)ethoxy]benzyl}-1-methyl-1H-pirazole twice a day) by oral route for improving urge incontinence caused by overactive bladder, by showing statistical and clinically significant improvements compared to placebo in the majority of efficacy variables considered.

### Example 3

Clinical Study B:

In a placebo controlled clinical trial 135 patients were randomised. Patients from both genders aged between 18 and 80 years (both inclusive) were included. They had Urinary incontinence secondary to overactive bladder (detrusor hyperreflexia or instability) or idiopathic urge incontinence confirmed by the medical history and urodynamic study.

The patients were treated within one group with (+)-5-{α-[2-(dimethylamino)ethoxy]benzyl}-1-methyl-1H-pirazole citrate, 230 mg b.i.d, capsules (meaning 400 mg (±)-5-{α-[2-(dimethylamino)ethoxy]benzyl}-1-methyl-1H-pirazole citrate/capsule) for oral administration (twice daily) or within another group with placebo, matching capsules, three per day (morning, afternoon and evening), for administration by oral route. The patients were treated for 84 days.

Efficacy was measured by the difference from baseline in the mean number of voidings/24 hours as provided by a 7-day frequency-volume chart in the end of study visit.

The primary efficacy analysis was based on the PP population. The treatment groups were compared with respect to the treatment effect, defined as the difference between treatment groups for changes from baseline in the number of voidings per 24 hours.

Treatment with (±)-5-{α-[2-(dimethylamino)ethoxy]benzyl}-1-methyl-1H-pirazole reduced the mean number of voidings per 24 hours in 5.83 units while placebo achieved a reduction of 2.39 units. The difference between them was statistically significant (95% confidence interval for the difference: −5.60, −1.28).

As an addition the percentage of responders was compared based on an analysis of the number of patients having <8 voidings/day or experienced complete dryness or both. The statistical significance was determined. (±)-5-{α-[2-(dimethylamino)ethoxy]benzyl}-1-methyl-1H-pirazole was superior to placebo (see FIG. 2).

The potential of (±)-5-{α-[2-(dimethylamino)ethoxy]benzyl}-1-methyl-1H-pirazole (230 mg b.i.d. by oral route) to improve bladder overactivity has been properly demonstrated in this clinical trial.

### Example 4

Example of Formulation for an Injectable (im/iv) Solution:

| | |
|---|---|
| Citrate of (±)-5-{α-[2- | 50 mg |
| (dimethylamino)ethoxy]benzyl}- | |
| 1-methyl-1H-pirazole | |
| 0.1 N Sodium hydroxide | c.s. pH 6 |
| Water for injection c.s.p. | 1 ml |

### Example 5

Example of a Formulation (A) for a Tablet

| | |
|---|---|
| (±)-5-{α-[2-(dimethylamino)ethoxy]benzyl}- | 400 mg |
| 1-methyl-1H-pirazole citrate | |
| Sodium croscarmelose (Ac-Di-Sol) | 32 mg |
| Colloidal silica dioxide (Aerosyl 200) | 8 mg |
| Magnesium stearate, NF | 16 mg |
| Povidone K-30 | 40 mg |
| Microcrystalline cellulose (Avicel PH-102) | 146 mg |
| Lactose monohydrate (Farmatose 200M) | 158 mg |
| Total | 800 mg |

### Example 6

Example of a Formulation (B) for a Tablet

| | |
|---|---|
| (±)-5-{α-[2-(dimethylamino)ethoxy]benzyl}- | 200 mg |
| 1-methyl-1H-pirazole citrate | |
| Sodium croscarmelose (Ac-Di-Sol) | 32 mg |
| Colloidal silica dioxide (Aerosyl 200) | 8 mg |
| Magnesium stearate, NF | 16 mg |
| Povidone K-30 | 40 mg |
| Microcrystalline cellulose (Avicel PH-102) | 246 mg |
| Lactose monohydrate (Farmatose 200M) | 258 mg |
| Total | 800 mg |

### Example 7

Example of a Formulation (C) for a Tablet

| | |
|---|---|
| (±)-5-{α-[2-(dimethylamino)ethoxy]benzyl}- | 400 mg |
| 1-methyl-1H-pirazole citrate | |
| Sodium croscarmelose (Ac-Di-Sol) | 35 mg |
| Colloidal silica dioxide (Aerosyl 200) | 3 mg |
| Sodium stearic fumarate | 12 mg |
| Polyethylene glycol 8000 | 30 mg |
| Microcrystalline cellulose (Avicel PH-102) | 75 mg |
| Lactose monohydrate (Farmatose 200M) | 45 mg |
| Total | 600 mg |

### Example 8

Example of a Formulation of a Capsule

| | |
|---|---|
| (±)-5-{α-[2-(dimethylamino)ethoxy]benzyl}- | 200.0 mg |
| 1-methyl-1H-pirazole citrate | |
| Colloidal silica dioxide | 0.8 mg |
| Magnesium stearate | 2.4 mg |
| Lactose | 276.8 mg |
| Total | 480 mg |

### Example 9

Actions of Cyclophosphamide

Cyclophosphamide is an effective form of treatment for several diseases including cancer. One possible side effect of this product is acute inflammation of the bladder. Its activity is based on conversion of the active metabolite in the liver. Treatment with cyclosphosphamide can give rise to several complications of adverse effects including urinary bladder cystitis, that is mainly due to another cyclophosphamide metabolite, acroleine. It is known that cyclophosphamide-induced cystitis is due to direct contact of acroleine with the urothelium, although the precise mechanism of this inflammatory response is largely unknown. One of the manifestations of inflammatory response is extravasation of plasma in the urinary bladder. Extravasation of plasmatic proteins has been measured by the permeability technique using Evan's blue dye, described by A. Saria and J. M. Lundberg (J. Neurosci. Methods 8: 41-49, 1983).

1. Use of an aryl derivative (or heteroaryl)azolylcarbinole of general formula (I) 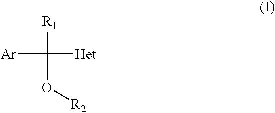
in which
Ar represents a phenyl radical or a thienyl radical, with no substitutions or optionally with 1, 2 or 3 equal or different substituents, selected from the group comprised of fluoride, chloride, bromide, methyl, trifluoromethyl and methoxy;
R₁ represents a hydrogen atom or a lower alkyl group from C, to C₄;
R₂ represents a dialkyl(C₁-C₄)aminoalkyl (C₂-C₃), or azaheterocyclylalkyl (C₂-C₃) radical; and
Het represents a five-armed nitrogenated aromatic heterocycle that contains one to three nitrogen atoms, without substitutions or optionally substituted by 1 or 2 equal or different substituents selected from a group comprised by fluoride, chloride, bromide and methyl;
or one of its physiologically acceptable salts,
in the production of a drug to treat urinary incontinence, in mammals, and also in man.

2. Use, according to claim 1, of a compound of general formula (I), in which R₁ is selected from a hydrogen atom or from the group comprised by methyl, ethyl, propyl, isopropyl, butyl, isobutyl, secbutyl and tert-butyl, in the production of a drug for the treatment of urinary incontinence, in mammals, including man.

3. Use, according to claim 1, of a compound of general formula (I), in which R₂ is selected from among a group comprised of dimethylaminoethyl, dimethylaminopropyl, diethylaminoethyl, piperidinylethyl, morpholinylpropyl and pirrolidinylethyl, in the production of a drug to treat urinary incontinence, in mammals, including man.

4. Use, according to claim 1, of a compound of general formula (I) selected from among a group comprised by:
(±)-5-{α-[2-(dimethylamino)ethoxy]benzyl}-1-methyl-1H-pirazole.
(±)-5-{α-[2-(dimethylamino)ethoxy]benzyl}-1-methyl-1H-pirazole citrate.
(+)-5-{α-[2-(dimethylamino)ethoxy]benzyl}-1-methyl-1H-pirazole.
(−)-5-{α-[2-(dimethylamino)ethoxy]benzyl}-1-methyl-1H-pirazole.
(+)-5-{α-[2-(dimethylamino)ethoxy]benzyl}-1-methyl-1H-pirazole citrate.
(−)-5-{α-[2-(dimethylamino)ethoxy]benzyl}-1-methyl-1H-pirazole citrate.
(±)-5-{α-[2-(dimethylamino)ethoxy]-2-thienylmethyl}-1-methyl-1H-pirazole.
(±)-5-{α-[2-(dimethylamino)ethoxy]-2-thienylmethyl}-1-methyl-1H-pirazole citrate.
(+)-5-{α-[2-(dimethylamino)ethoxy]-2-thienylmethyl}-1-methyl-1H-pirazole
(−)-5-{α-[2-(dimethylamino)ethoxy]-2-thienylmethyl}-1-methyl-1H-pirazole.
(+)-5-{α-[2-(dimethylamino)ethoxy]-2-thienylmethyl}-1-methyl-1H-pirazole citrate.
(−)-5-{α-[2-(dimethylamino)ethoxy]-2-thienylmethyl}-1-methyl-1H-pirazole citrate,
in the production of a drug to treat urinary incontinence, in mammals, including man.

5. A pharmaceutical composition, characterised because it contains, at least, one compound of general formula (I) or one of its physiologically acceptable salts, according to claim 1, and the pharmaceutically acceptable excipients, to treat urinary incontinence, in mammals, including man.

6. A pharmaceutical composition, characterised because it contains, at least, one compound of general formula (I) or one of its physiologically acceptable salts, according to claim 2, and the pharmaceutically acceptable excipients, to treat urinary incontinence, in mammals, including man.

7. A pharmaceutical composition, characterised because it contains, at least, one compound of general formula (I) or one of its physiologically acceptable salts, according to claim 3, and the pharmaceutically acceptable excipients, to treat urinary incontinence, in mammals, including man.

8. A pharmaceutical composition, characterised because it contains, at least, one compound of general formula (I) or one of its physiologically acceptable salts, according to claim 4, and the pharmaceutically acceptable excipients, to treat urinary incontinence, in mammals, including man.

9. Method of treatment of a patient or a mammal, including man, suffering from urinary incontinence characterized in that the method comprises the administration of a therapeutically effective amount of a compound of general formula (I) 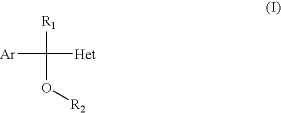
in which
Ar represents a phenyl radical or a thienyl radical, with no substitutions or optionally with 1, 2 or 3 equal or different substituents, selected from a group consisting of fluoride, chloride, bromide, methyl, trifluoromethyl and methoxy;
R₁ represents hydrogen or a lower alkyl group from C₁ to C₄;
R₂ represents a dialkyl(C₁-C₄)aminoalkyl (C₂-C₃), or azaheterocyclylalkyl (C₂-C₃) radical; and
Het represents a five-armed nitrogenated aromatic heterocycle that contains one to three nitrogen atoms, without substitutions or optionally substituted by 1 or 2 equal or different substituents selected from a group consisting of fluoride, chloride, bromide and methyl;
optionally in the form of its racemate, pure stereoisomers, especially enantiomers or diastereomers or in the form of mixtures of stereoisomers, especially enantiomers or diastereomers, in any suitable ratio;
in the form shown or in form of the acid or base or in form of a salt, especially a physiologically acceptable salt, or in form of a solvate, especially a hydrate.

10. Method, according to claim 9, characterized in that it comprises the administration of a compound of general formula (I), in which R₁ is selected from hydrogen or from a group consisting of methyl, ethyl, propyl, isopropyl, butyl, isobutyl, sec-butyl and tert-butyl.

11. Method, according to claim 9, characterized in that it comprises the administration of a compound of general formula (I), in which R₂ is selected from among a group consisting of dimethylaminoethyl, dimethylaminopropyl, diethylaminoethyl, piperidinylethyl, morpholinylpropyl and pirrolidinylethyl.

12. Method according to claim 9, characterized in that it comprises the administration of a compound of general formula (Ia) 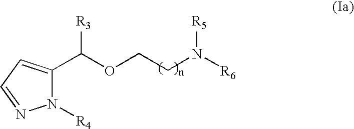
in which
n is 1 or 2;
R₃ is selected from: 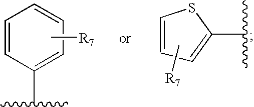
R₄ is selected from hydrogen, fluoride, chloride, bromide and methyl;
R₅ and R₆ are independently selected from lower C₍₁₋₄₎-Alkyl or together with the Nitrogen form an azaheterocyclic ring;
R₇ is selected from the group consisting of hydrogen, fluoride, chloride, bromide, methyl, trifluoromethyl and methoxy.

13. Method, according to claim 12, characterized in that it comprises the administration of a compound of general formula (Ia), in which R₇ is hydrogen.

14. Method, according to claim 12, characterized in that it comprises the administration of a compound of general formula (Ia), in which R₄ is Methyl.

15. Method, according to claim 12, characterized in that it comprises the administration of a compound of general formula (Ia), in which R₄ and R₅ are either CH₃ or C₂H₅ or together with the Nitrogen form a piperidinyl, morpholinyl or pirrolidinyl ring.

16. Method, according to claim 12, characterized in that it comprises the administration of a compound of general formula (Ia) selected from among a group consisting of:
5-{α-[2-(dimethylamino)ethoxy]benzyl}-1-methyl-1H-pirazole,
(±)-5-{α-[2-(dimethylamino)ethoxy]benzyl}-1-methyl-1H-pirazole,
(+)-5-{α-[2-(dimethylamino)ethoxy]benzyl}-1-methyl-1H-pirazole,
(−)-5-{α-[2-(dimethylamino)ethoxy]benzyl}-1-methyl-1H-pirazole,
5-{α-[2-(dimethylamino)ethoxy]benzyl}-1-methyl-1H-pirazole citrate,
(±)-5-{α-[2-(dimethylamino)ethoxy]benzyl}-1-methyl-1H-pirazole citrate,
(+)-5-{α-[2-(dimethylamino)ethoxy]benzyl}-1-methyl-1H-pirazole citrate,
(−)-5-{α-[2-(dimethylamino)ethoxy]benzyl}-1-methyl-1H-pirazole citrate,
5-{α-[2-(dimethylamino)ethoxy]-2-thienylmethyl}-1-methyl-1H-pirazole,
(±)-5-{α-[2-(dimethylamino)ethoxy]-2-thienylmethyl}-1-methyl-1H-pirazole,
(+)-5-{α-[2-(dimethylamino)ethoxy]-2-thienylmethyl}-1-methyl-1H-pirazole,
(−)-5-{α-[2-(dimethylamino)ethoxy]-2-thienylmethyl}-1-methyl-1H-pirazole,
5-{α-[2-(dimethylamino)ethoxy]-2-thienylmethyl}-1-methyl-1H-pirazole citrate,
(±)-5-{α-[2-(dimethylamino)ethoxy]-2-thienylmethyl}-1-methyl-1H-pirazole citrate,
(+)-5-{α-[2-(dimethylamino)ethoxy]-2-thienylmethyl}-1-methyl-1H-pirazole citrate,
(−)-5-{α-[2-(dimethylamino)ethoxy]-2-thienylmethyl}-1-methyl-1H-pirazole citrate.

17. Method according to claim 12, characterized in that it comprises the administration of a compound of general formula (Ib) 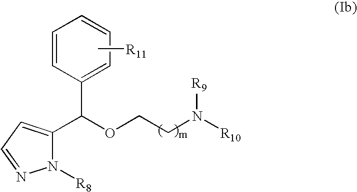
in which
m is 1 or 2;
R₈ is selected from hydrogen, fluoride, chloride, bromide and methyl;
R₉ and R₁₀ are independently selected from lower C₍₁₋₄₎-Alkyl or together with the Nitrogen form an azaheterocyclic ring;
R₁₁ is selected from the group consisting of hydrogen, fluoride, chloride, bromide, methyl, trifluoromethyl and methoxy.

18. Method, according to claim 17, characterized in that it comprises the administration of a compound of general formula (Ib), in which R₁₁ is hydrogen.

19. Method, according to claim 17, characterized in that it comprises the administration of a compound of general formula (Ib), in which R₈ is Methyl.

20. Method, according to claim 17, characterized in that it comprises the administration of a compound of general formula (Ib), in which R₉ and R₁₀ are either CH₃ or C₂H₅ or together with the Nitrogen form a piperidinyl, morpholinyl or pirrolidinyl ring;
preferably in which R₉ and R₁₀ are either CH₃ or C₂H₅;
especially in which R₉ and R₁₀ are equal and either CH₃ or C₂H₅;
most preferably in which R₉ and R₁₀ are both CH₃.

21. Method, according to claim 17, characterized in that it comprises the administration of a compound of general formula (Ib), in which m is 1.

22. Method, according to claim 17, characterized in that it comprises the administration of a compound of general formula (Ib) selected from among a group consisting of:
5-{α-[2-(dimethylamino)ethoxy]benzyl}-1-methyl-1H-pirazole,
(±)-5-{α-[2-(dimethylamino)ethoxy]benzyl}-1-methyl-1H-pirazole,
(+)-5-{α-[2-(dimethylamino)ethoxy]benzyl}-1-methyl-1H-pirazole,
(−)-5-{α-[2-(dimethylamino)ethoxy]benzyl}-1-methyl-1H-pirazole,
5-{α-[2-(dimethylamino)ethoxy]benzyl}-1-methyl-1H-pirazole citrate,
(±)-5-{α-[2-(dimethylamino)ethoxy]benzyl}-1-methyl-1H-pirazole citrate,
(+)-5-{α-[2-(dimethylamino)ethoxy]benzyl}-1-methyl-1H-pirazole citrate,
(−)-5-{α-[2-(dimethylamino)ethoxy]benzyl}-1-methyl-1H-pirazole citrate.

23. Method, according to claim 17, characterized in that it comprises the administration of
5-{α-[2-(dimethylamino)ethoxy]benzyl}-1-methyl-1H-pirazole.

24. Method, according to claim 17, characterized in that it comprises the administration of
(±)-5-{α-[2-(dimethylamino)ethoxy]benzyl}-1-methyl-1H-pirazole.

25. Method, according to claim 17, characterized in that it comprises the administration of
(+)-5-{α-[2-(dimethylamino)ethoxy]benzyl}-1-methyl-1H-pirazole.

26. Method, according to claim 17, characterized in that it comprises the administration of
(−)-5-{α-[2-(dimethylamino)ethoxy]benzyl}-1-methyl-1H-pirazole.

27. Method, according to claim 17, characterized in that it comprises the administration of
5-{α-[2-(dimethylamino)ethoxy]benzyl}-1-methyl-1H-pirazole citrate.

28. Method, according to claim 17, characterized in that it comprises the administration of
(±)-5-{α-[2-(dimethylamino)ethoxy]benzyl}-1-methyl-1H-pirazole citrate.

29. Method, according to claim 17, characterized in that it comprises the administration of
(+)-5-{α-[2-(dimethylamino)ethoxy]benzyl}-1-methyl-1H-pirazole citrate.

30. Method, according to claim 17, characterized in that it comprises the administration of
(−)-5-{α-[2-(dimethylamino)ethoxy]benzyl}-1-methyl-1H-pirazole citrate.

31. Method according to claim 12, characterized in that it comprises the administration of a compound of general formula (Ic) 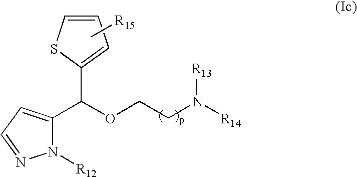
in which
p is 1 or 2;
R₁₂ is selected from hydrogen, fluoride, chloride, bromide and methyl;
R₁₃ and R₁₄ are independently selected from lower C₍₁₋₄₎-Alkyl or together with the Nitrogen form an azaheterocyclic ring;
R₁₅ is selected from the group consisting of hydrogen, fluoride, chloride, bromide, methyl, trifluoromethyl and methoxy.

32. Method, according to claim 31, characterized in that it comprises the administration of a compound of general formula (Ic), in which R₁₅ is hydrogen.

33. Method, according to claim 31, characterized in that it comprises the administration of a compound of general formula (Ic), in which R₁₂ is Methyl.

34. Method, according to claim 31, characterized in that it comprises the administration of a compound of general formula (Ic), in which R₁₃ and R₁₄ are either CH₃ or C₂H₅ or together with the Nitrogen form a piperidinyl, morpholinyl or pirrolidinyl ring;
preferably in which R₁₃ and R₁₄ are either CH₃ or C₂H₅;
especially in which R₁₃ and R₁₄ are equal and either CH₃ or C₂H₅;
most preferably in which R₁₃ and R₁₄ are both CH₃.

35. Method, according to claim 31, characterized in that it comprises the administration of a compound of general formula (Ic), in which p is 1.

36. Method, according to claim 31, characterized in that it comprises the administration of a compound of general formula (Ic) selected from among a group consisting of:
5-{α-[2-(dimethylamino)ethoxy]-2-thienylmethyl}-1-methyl-1H-pirazole,
(±)-5-{α-[2-(dimethylamino)ethoxy]-2-thienylmethyl}-1-methyl-1H-pirazole,
(+)-5-{α-[2-(dimethylamino)ethoxy]-2-thienylmethyl}-1-methyl-1H-pirazole,
(−)-5-{α-[2-(dimethylamino)ethoxy]-2-thienylmethyl}-1-methyl-1H-pirazole,
5-{α-[2-(dimethylamino)ethoxy]-2-thienylmethyl}-1-methyl-1H-pirazole citrate,
(±)-5-{α-[2-(dimethylamino)ethoxy]-2-thienylmethyl}-1-methyl-1H-pirazole citrate,
(+)-5-{α-[2-(dimethylamino)ethoxy]-2-thienylmethyl}-1-methyl-1H-pirazole citrate,
(−)-5-{α-[2-(dimethylamino)ethoxy]-2-thienylmethyl}-1-methyl-1H-pirazole citrate.

37. Method, according to claim 31, characterized in that it comprises the administration of:
5-{α-[2-(dimethylamino)ethoxy]-2-thienylmethyl}-1-methyl-1H-pirazole,
,optionally in the form of its racemate, pure stereoisomers, especially enantiomers or diastereomers or in the form of mixtures of stereoisomers, especially enantiomers or diastereomers, in any suitable ratio;
in form of the free base or in form of a salt, especially a physiologically acceptable salt, or in form of a solvate, especially a hydrate.

38. Method, according to claim 31, characterized in that it comprises the administration of:
5-{α-[2-(dimethylamino)ethoxy]-2-thienylmethyl}-1-methyl-1H-pirazole citrate,
,optionally in the form of its racemate, pure stereoisomers, especially enantiomers or diastereomers or in the form of mixtures of stereoisomers, especially enantiomers or diastereomers, in any suitable ratio.

39. Method, according to claim 9, characterized in that man means a female.

40. Method, according to claim 9, characterized in that man means a male.

41. Method, according to claim 9, characterized in that the patient is a woman.

42. Method, according to claim 9, characterized in that the patient is an elderly woman.

43. Method, according to claim 9, characterized in that the patient is a man.

44. Method, according to claim 9, characterized in that the patient is an elderly man.

45. Method, according to claim 9, characterized in that the patient is a child.

46. Method, according to claim 9, characterized in that the urinary incontinence the patient or mammal, including man, is suffering from is urge urinary incontinence.

47. Method, according to claim 9, characterized in that the urinary incontinence the patient or mammal, including man, is suffering from is stress urinary incontinence or urinary stress incontinence.

48. Method, according to claim 9, characterized in that the urinary incontinence the patient or mammal, including man, is suffering from is hyperreflexive urinary incontinence.

49. Method, according to claim 9, characterized in that the urinary incontinence the patient or mammal, including man, is suffering from is enuresis.

50. Method according to claim 9 characterized in that the therapeutically effective amount of the active compound is administered at a dose between 50 and 400 mg/day or between 200 and 600 mg/day.

51. Method according to claim 9 characterized in that the compound is administered in form of a tablet or capsule.

52. Method according to claim 9 characterized in that the compound is administered in form of an immediate release formulation.

53. Method of treatment of a patient suffering from urinary incontinence characterized in that the method comprises the administration of a therapeutically effective amount of
5-{α-[2-(dimethylamino)ethoxy]benzyl}-1-methyl-1H-pirazole,
,optionally in the form of its racemate, pure stereoisomers, especially enantiomers or diastereomers or in the form of mixtures of stereoisomers, especially enantiomers or diastereomers, in any suitable ratio;
in form of the free base or in form of a salt, especially a physiologically acceptable salt, or in form of a solvate, especially a hydrate.

54. Method, according to claim 53, characterized in that it comprises the administration of
(±)-5-{α-[2-(dimethylamino)ethoxy]benzyl}-1-methyl-1H-pirazole.

55. Method, according to claim 53, characterized in that it comprises the administration of
(+)-5-{α-[2-(dimethylamino)ethoxy]benzyl}-1-methyl-1H-pirazole.

56. Method, according to claim 53, characterized in that it comprises the administration of
(−)-5-{α-[2-(dimethylamino)ethoxy]benzyl}-1-methyl-1H-pirazole.

57. Method, according to claim 53, characterized in that it comprises the administration of
5-{α-[2-(dimethylamino)ethoxy]benzyl}-1-methyl-1H-pirazole citrate.

58. Method, according to claim 53, characterized in that it comprises the administration of
(±)-5-{α-[2-(dimethylamino)ethoxy]benzyl}-1-methyl-1H-pirazole citrate.

59. Method, according to claim 53, characterized in that it comprises the administration of
(+)-5-{α-[2-(dimethylamino)ethoxy]benzyl}-1-methyl-1H-pirazole citrate.

60. Method, according to claim 53, characterized in that it comprises the administration of
(−)-5-{α-[2-(dimethylamino)ethoxy]benzyl}-1-methyl-1H-pirazole citrate.

61. Method, according to claim 53, characterized in that the patient is a woman.

62. Method, according to claim 53, characterized in that the patient is an elderly woman.

63. Method, according to claim 53, characterized in that the patient is a man.

64. Method, according to claim 53, characterized in that the patient is an elderly man.

65. Method, according to claim 53, characterized in that the patient is a child.

66. Method, according to claim 53, characterized in that the urinary incontinence the patient is suffering from is urge urinary incontinence.

67. Method, according to claim 53, characterized in that the urinary incontinence the patient is suffering from is stress urinary incontinence or urinary stress incontinence.

68. Method, according to claim 53, characterized in that the urinary incontinence the patient is suffering from is hyperreflexive urinary incontinence.

69. Method, according to claim 53, characterized in that the urinary incontinence the patient is suffering from is enuresis.

70. Method according to claim 53 characterized in that the therapeutically effective amount of 5-{α-[2-(dimethylamino)ethoxy]benzyl}-1-methyl-1H-pirazole is administered at a dose between 50 and 400 mg/day or between 200 and 600 mg/day.

71. Method according to claim 53 characterized in that the therapeutically effective amount of (±)-5-{α-[2-(dimethylamino)ethoxy]benzyl}-1-methyl-1H-pirazole is administered at a dose between 50 and 400 mg/day or between 200 and 600 mg/day.

72. Method according to claim 53 characterized in that the therapeutically effective amount of (+)-5-{α-[2-(dimethylamino)ethoxy]benzyl}-1-methyl-1H-pirazole is administered at a dose between 50 and 400 mg/day or between 200 and 600 mg/day.

73. Method according to claim 53 characterized in that the therapeutically effective amount of (−)-5-{α-[2-(dimethylamino)ethoxy]benzyl}-1-methyl-1H-pirazole is administered at a dose between 50 and 400 mg/day or between 200 and 600 mg/day.

74. Method according to claim 53 characterized in that 5-{α-[2-(dimethylamino)ethoxy]benzyl}-1-methyl-1H-pirazole is administered at a dose of 230 mg/day, 460 mg/day or 345 mg/day.

75. Method according to claim 53 characterized in that (±)-5-{α-[2-(dimethylamino)ethoxy]benzyl}-1-methyl-1H-pirazole is administered at a dose of 230 mg/day, 345 mg/day, 460 mg/day or 575 mg/day, preferably 345 mg/day or 460 mg/day.

76. Method according to claim 53 characterized in that 5-{α-[2-(dimethylamino)ethoxy]benzyl}-1-methyl-1H-pirazole citrate is administered at a dose of 400 mg/day, 600 mg/day, 800 mg/day or 1000 mg/day, preferably 600 mg/day or 800 mg/day.

77. Method according to claim 53 characterized in that (±)-5-{α-[2-(dimethylamino)ethoxy]benzyl}-1-methyl-1H-pirazole citrate is administered at a dose of 400 mg/day, 600 mg/day, 800 mg/day or 1000 mg/day, preferably 600 mg/day or 800 mg/day.

78. Method according to claim 53 characterized in that 5-{α-[2-(dimethylamino)ethoxy]benzyl}-1-methyl-1H-pirazole is administered twice daily.

79. Method according to claim 53 characterized in that 5-{α-[2-(dimethylamino)ethoxy]benzyl}-1-methyl-1H-pirazole is administered orally.

80. Method according to claim 53 characterized in that 5-{α-[2-(dimethylamino)ethoxy]benzyl}-1-methyl-1H-pirazole is administered in form of a tablet or capsule.

81. Method according to claim 53 characterized in that 5-{α-[2-(dimethylamino)ethoxy]benzyl}-1-methyl-1H-pirazole is administered in form of an immediate release formulation.

82. Method according to claim 53 characterized in that 5-{α-[2-(dimethylamino)ethoxy]benzyl}-1-methyl-1H-pirazole is administered in form of a formulation comprising any of the following:
sodium croscarmelose
colloidal silica dioxide,
a salt with stearic acid, especially magnesium stearate,
povidone,
microcrystalline cellulose
lactose monohydrate
polyethylene glycol.

83. Method according to claim 53 characterized in that 5-{α-[2-(dimethylamino)ethoxy]benzyl}-1-methyl-1H-pirazole is administered in form of a formulation according to example 5.

84. Method according to claim 53 characterized in that 5-{α-[2-(dimethylamino)ethoxy]benzyl}-1-methyl-1H-pirazole is administered in form of a formulation according to example 7.
